(19) United States
(12) Patent Application Publication
(10) Pub. No.: US 2024/0051916 A1
(43) Pub. Date: Feb. 15, 2024

(54) METHOD FOR PRODUCING N-(HETERO)ARYL (METH)ACRYLAMIDE COMPOUND

(71) Applicant: FUJIFILM Corporation, Tokyo (JP)

(72) Inventors: Yasutaka TASAKI, Kanagawa (JP); Kenji WADA, Kanagawa (JP)

(73) Assignee: FUJIFILM Corporation, Tokyo (JP)

(21) Appl. No.: 18/488,978

(22) Filed: Oct. 17, 2023

Related U.S. Application Data
(63) Continuation of application No. PCT/JP2022/021000, filed on May 20, 2022.

(30) Foreign Application Priority Data
May 28, 2021 (JP) 2021-089874
Nov. 19, 2021 (JP) 2021-188898
Mar. 23, 2022 (JP) 2022-046961

(51) Int. Cl. C07C 231/02

(52) U.S. Cl. CPC C07C 231/02

(57) ABSTRACT

Provided is a method for producing an N-(hetero)aryl (meth)acrylamide compound, including reacting a compound represented by General Formula (1) with a compound represented by General Formula (2) at a temperature higher than 120° C. to carry out amidation and to obtain a compound represented by General Formula (3).

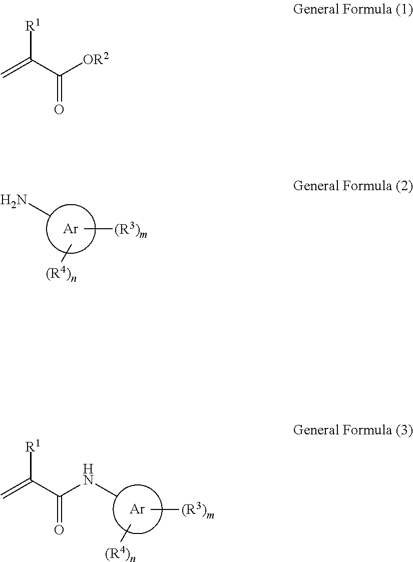

R¹ represents a hydrogen atom or an aliphatic group, R² and R⁴ represent a hydrogen atom, a chain-like aliphatic group, an aliphatic hydrocarbon ring group, an aryl group, or a heterocyclic group, Ar represents an aromatic ring, R³ represents an electron withdrawing group, m represents an integer of 1 or more, and n represents an integer of 0 or more. R⁴ is not an α-hydroxybenzyl group.

### CROSS-REFERENCE TO RELATED APPLICATIONS

This application is a Continuation of PCT International Application No. PCT/JP2022/021000 filed on May 20, 2022, which claims priority under 35 U.S.C. § 119 (a) to Japanese Patent Application No. 2021-089874 filed in Japan on May 28, 2021, Japanese Patent Application No. 2021-188898 filed in Japan on Nov. 19, 2021, and Japanese Patent Application No. 2022-046961 filed in Japan on Mar. 23, 2022. Each of the above applications is hereby expressly incorporated by reference, in its entirety, into the present application.

### BACKGROUND OF THE INVENTION

### 1. Field of the Invention

The present invention relates to a method for producing an N-(hetero)aryl (meth)acrylamide compound.

### 2. Description of the Related Art

An N-(hetero)aryl (meth)acrylamide compound has been used in various fields as a highly functional polymer. For example, 4-sulfamoylphenyl methacrylamide is used in a drug delivery system for medical use, and is also used in a lithographic printing plate in order to improve solvent resistance, alkali resistance, and the like.

The N-(hetero)aryl (meth)acrylamide compound can be obtained by reacting a (meth)acrylic acid compound with an N-(hetero)arylamine compound (amidation reaction). Various methods have already been proposed for the amidation reaction itself, and for example, a symmetric acid anhydride method, a mixed acid anhydride method, an acid chloride method, a condensing agent method, an amine activation method, and the like are known.

In the symmetric acid anhydride method, (meth)acrylic acid is activated to be an anhydride, and this (meth)acrylic acid anhydride is reacted with an N-(hetero)arylamine compound to obtain the N-(hetero)aryl (meth)acrylamide compound (for example, CN103467346C). In the (meth)acrylic acid anhydride used in the reaction, one of two (meth)acrylic acid components constituting the anhydride is (meth)acrylic acid which is a by-product. As a result, it is necessary to separate and remove a large amount of by-produced (meth)acrylic acid which causes a heavy environmental load. In addition, the (meth)acrylic acid anhydride is a relatively expensive reagent, and thus there is a limit in terms of cost.

In the mixed acid anhydride method, (meth)acrylic acid is reacted with, for example, chloroformic acid ester to prepare a mixed acid anhydride which is an activated form of (meth)acrylic acid. By reacting the mixed acid anhydride with an N-(hetero)arylamine compound, an N-aryl (meth)acrylamide compound is obtained (for example, JP2008-151929A). In the mixed acid anhydride method, the chloroformic acid ester is a by-product. Therefore, it is necessary to separate and remove a large amount of by-products after the reaction, which is also a method with a large environmental load.

In the acid chloride method, (meth)acryloyl chloride which is an activated form of (meth)acrylic acid is reacted with an N-(hetero)arylamine compound to obtain the N-(hetero)aryl (meth)acrylamide compound (for example, JP1974-010499B (JP-S49-010499B)). The (meth)acryloyl chloride is expensive, and the acid chloride method has a restriction in terms of cost.

In the condensing agent method, (meth)acrylic acid is activated by a condensing agent, and reacted with an N-(hetero)arylamine compound to obtain the N-(hetero)aryl (meth)acrylamide compound (for example, US2005/0107341A). The condensing agent is generally an expensive reagent, and it is necessary to separate and remove a residue of the condensing agent after the reaction. As a result, an operation is complicated and environmental load is also increased.

In the amine activation method, an anion is generated on an amino group of an N-(hetero)arylamine compound by using an organic metal reagent such as n-butyl lithium to activate the compound, and then the compound is reacted with a (meth)acrylic acid compound to obtain the N-(hetero)aryl (meth)acrylamide compound. Many organic metal reagents are water-resistant and may ignite, and thus the reaction needs to be carried out at an extremely low temperature. Therefore, the method is difficult to scale up to an industrial production level.

In addition, the reaction between the (meth)acrylic acid compound and the N-(hetero)arylamine compound causes the following problem in addition to the above-described problems. That is, there is a problem that, other than the desired N-(hetero)aryl (meth)acrylamide compound (1,2-adduct), a large amount of by-products (1,4-adducts) are produced by a reaction of the N-(hetero)arylamine compound with a double bonding site of the (meth)acrylic acid compound. In order to solve this problem, it is necessary to increase regioselectivity of the reaction between the (meth)acrylic acid compound and the N-(hetero)arylamine compound. In order to deal with this problem, JP1979-138513A (JP-S54-138513A) proposes performing the reaction in the presence of a catalytic amount of dialkyl tin oxide.

In addition, CN109608367C proposes activating (meth)acrylic acid ester with a Lewis acid to synthesize an N-aryl (meth)acrylamide compound.

### SUMMARY OF THE INVENTION

As described above, in order to obtain the N-(hetero)aryl (meth)acrylamide compound by the reaction between the (meth)acrylic acid compound and the N-(hetero)arylamine compound with high efficiency, it is important to suppress the production of 1,4-adduct which is a by-product. As a result of studies by the present inventors, it has been found that, in a case where an N-(hetero)arylamine compound has a structure in which ring-constituting atoms of an aromatic ring have an electron withdrawing group as a substituent (for example, sulfanylamide), the problem of regioselectivity of the reaction is more actualized, and the amount of 1,4-adducts produced far exceeds the amount of the desired 1,2-adducts produced. As an N-(hetero)aryl (meth)acrylamide compound obtained from the N-(hetero)arylamine compound having such an electron withdrawing group, industrially important compounds have been known such as the 4- sulfamoylphenyl methacrylamide described above.

An object of the present invention is to provide a method for producing an N-(hetero)aryl (meth)acrylamide compound that, in production of an N-(hetero)aryl (meth)acrylamide compound, which includes reacting a (meth)acrylic acid compound with an N-(hetero)arylamine compound, while using, as the raw material of N-(hetero)arylamine compound, an N-(hetero)arylamine compound in which ring-constituting atoms of an aromatic ring have an electron withdrawing group as a substituent, a formation of 1,4-adducts as by-products can be sufficiently suppressed, a target N-(hetero)aryl (meth)acrylamide compound can be obtained with high selectivity, and it is also possible to appropriately reduce cost and environmental load resulting from raw materials and reagents.

As a result of intensive studies in consideration of the above-described object, the present inventors have found that, in reaction of the (meth)acrylic acid compound with the above-described N-(hetero)arylamine compound having an electron withdrawing group to obtain the N-(hetero)aryl (meth)acrylamide compound, the above-described object can be achieved by controlling a reaction temperature to a high temperature range of higher than 120° C. That is, it has been found that, in a chemical reaction, it is common to control the reaction temperature in a low temperature range in order to increase regioselectivity of the reaction; but by controlling the reaction temperature to a high temperature of higher than 120° C., the regioselectivity of the reaction can be dramatically increased without using the above-described expensive activator as a raw material or without using a special reagent such as the condensing agent. The present invention has been completed by further repeating studies on the basis of the above-described finding.

That is, the object of the present invention has achieved by the following methods.

[1]

A method for producing an N-(hetero)aryl (meth)acrylamide compound, the production method comprising:
reacting a compound represented by General Formula (1) with a compound represented by General Formula (2) at a temperature higher than 120° C. to carry out amidation and to obtain a compound represented by General Formula (3),

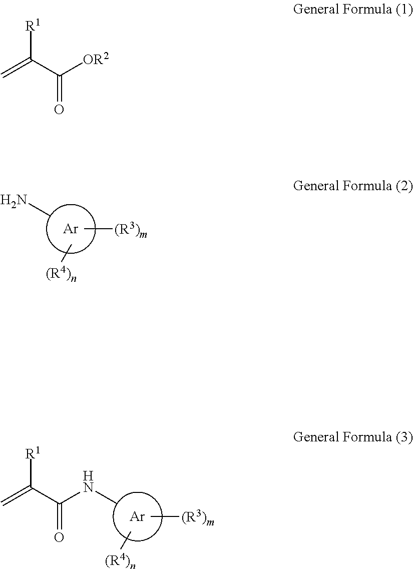

in each formula, R¹ represents a hydrogen atom or an aliphatic group, R² represents a hydrogen atom, a chain-like aliphatic group, an aliphatic hydrocarbon ring group, an aryl group, or a heterocyclic group, Ar represents an aromatic ring, R³ represents an electron withdrawing group and m is an integer of 1 or more, R⁴ represents a chain-like aliphatic group, an aliphatic hydrocarbon ring group, an aryl group, or a heterocyclic group, and n is an integer of 0 or more, where R⁴ is not an α-hydroxybenzyl group, and a maximum value of m+n is a maximum value of the number of substituents which can be included in ring-constituting atoms of Ar.

[2]

The production method according to [1],
in which the amidation reaction is carried out in a presence of at least one reaction catalyst.

[3]

The production method according to [2],
in which the reaction catalyst is at least one of a Lewis acid, a Broensted acid, a metal oxide, or a phosphorus oxide compound.

[4]

The production method according to [2],
in which at least one of a Lewis acid, a Broensted acid, or a phosphorus oxide compound is used as the reaction catalyst.

[5]

The production method according to any one of [1] to [4],
in which a reaction temperature of the amidation reaction is set to 130° C. or higher.

[6]

The production method according to [5],
in which the reaction temperature of the amidation reaction is set to 140° C. or higher.

[7]

The production method according to any one of [1] to [6],
in which Ar represents a benzene ring.

[8]

The production method according to [7],
in which R¹ represents a hydrogen atom or an alkyl group having 1 to 5 carbon atoms.

[9]

The production method according to [7] or [8],
in which m is an integer of 1 to 3 and n is an integer of 0 to 4.

[10]

The production method according to any one of [1] to [9],
in which a reaction temperature of the amidation reaction is controlled by micro wave irradiation.

[11]

The production method according to any one of [1] to [10],
in which the amidation reaction is carried out by a flow-type reaction.

In the present invention or the specification, any numerical range expressed using “to” refers to a range including the numerical values before and after the “to” as a lower limit value and an upper limit value, respectively.

In the present invention or the specification, with regard to a substituent in which whether it is substituted or unsubstituted is not specified, within a range not impairing the desired effect, it means that the group may have an optional substituent. The same is applied to a compound which is not specified regarding whether to be substituted or unsubstituted.

In the present invention or the specification, in a case of being simply referred to as “substituent”, it is preferable that a group selected from a substituent group Z described later can be applied. In addition, in a case where only the name of each group is described (for example, in a case where only “alkyl group” is described”), as its preferred form, the preferred range and specific examples of the corresponding group (alkyl group in the case) of the substituent group Z are applied.

In the present invention or the specification, in a case of defining a number of carbon atoms in a group, the number of carbon atoms means the number of carbon atoms in the entire group. That is, in a case where the group has a substituent, it means the total number of carbon atoms including the substituent.

In the present specification, a term “. . . compound” is intended to include a compound which has a common basic skeleton, but a structure thereof is partially changed within the scope of achieving the desired effect (for example, a compound in which some hydrogen atoms are substituted with substituents). For example, “(meth)acrylic acid compound” is intended to include, in addition to (meth)acrylic acid, a compound derived from (meth)acrylic acid to the extent that the desired effect is achieved, and “N-(hetero)aryl (meth)acrylamide compound” is intended to include, in addition to N-(hetero)aryl (meth)acrylamide, a compound derived from N-(hetero)aryl (meth)acrylamide to the extent that the desired effect is achieved.

In the present invention or the specification, “(meth)acrylic” is intended to include both structures of methacrylic and acrylic. For example, “(meth)acrylic acid compound” means a methacrylic acid compound and/or an acrylic acid compound. In addition, the term “methacrylic” is used to have a broader meaning than usual. That is, in a structure of “CH₂═C(R¹)CO—”, as defined in General Formula (1), the term “methacrylic” (methacryloyl) is used to indicate not only a form of methyl for R¹ but also all forms of aliphatic groups for R¹. According to the interpretation of the term “. . . compound” described above, the methacrylic acid compound can be considered to be included in the acrylic acid compound, but considering that the “(meth)acrylic” is a commonly used expression in the field of chemistry, the expression “(meth)acrylic” is used.

In the present invention or the specification, “(hetero)aryl” is intended to include both structures of heteroaryl (aromatic heterocyclic group) and aryl (aromatic hydrocarbon ring group).

According to the present invention, it is possible to, while using, as a raw material of N-(hetero)arylamine compound, an N-(hetero)arylamine compound in which ring-constituting atoms of an aromatic ring have an electron withdrawing group as a substituent, sufficiently suppress a formation of 1,4-adducts as by-products, and obtain a target N-(hetero)aryl (meth)acrylamide compound with high selectivity. According to the present invention, since it is not necessary to use an expensive activator or a special reagent in the production of the N-(hetero)aryl (meth)acrylamide compound, it is possible to appropriately reduce cost and environmental load resulting from raw materials and reagents.

### DESCRIPTION OF THE PREFERRED EMBODIMENTS

The object of the present invention is to provide a method for producing an N-(hetero)aryl (meth)acrylamide compound, including reacting a compound represented by General Formula (1) [(meth)acrylic acid compound] with a compound represented by General Formula (2) [N-(hetero)arylamine compound] at a temperature higher than 120° C. to carry out amidation and to obtain a compound represented by General Formula (3) [N-(hetero)aryl (meth)acrylamide compound] (hereinafter, also referred to as a production method according to the embodiment of the present invention).

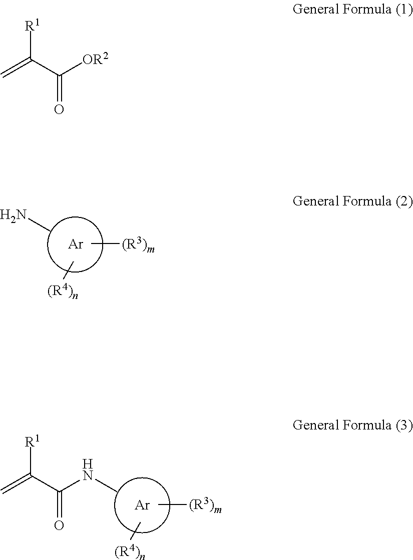

In General Formula (1), R¹ represents a hydrogen atom or an aliphatic group. The aliphatic group which can be adopted as R¹ may be a saturated aliphatic group or an unsaturated aliphatic group. The number of carbon atoms in the aliphatic group which can be adopted as R¹ is preferably 1 to 20, more preferably 1 to 18, still more preferably 1 to 15, still more preferably 1 to 12, still more preferably 1 to 10, still more preferably 1 to 8, still more preferably 1 to 6, and still more preferably 1 to 5. The aliphatic group which can be adopted as R¹ is preferably an aliphatic hydrocarbon group. The aliphatic hydrocarbon group is more preferably an alkyl group, an alkenyl group, or an alkynyl group.

The alkyl group which can be adopted as R¹ may be linear or branched, or may form a ring. The number of carbon atoms in the alkyl group is preferably 1 to 20 (in a case where the alkyl group has a ring structure (cycloalkyl group), the lower limit of the number of carbon atoms is 3, preferably 4 and more preferably 5; the same applies hereinafter), more preferably 1 to 18, still more preferably 1 to 15, still more preferably 1 to 12, still more preferably 1 to 10, still more preferably 1 to 8, still more preferably 1 to 6, and still more preferably 1 to 5. The alkyl group which can be adopted as R¹ is preferably an unsubstituted alkyl group or trifluoromethyl. The alkyl group which can be adopted as R¹ is more preferably methyl, trifluoromethyl, ethyl, propyl, or butyl, still more preferably methyl, trifluoromethyl, or ethyl, and particularly preferably methyl.

The alkenyl group which can be adopted as R¹ may be linear or branched, or may form a ring. The number of carbon atoms in the alkenyl group is preferably 2 to 20 (in a case where the alkenyl group has a ring structure (cycloalkenyl group), the lower limit of the number of carbon atoms is 3, preferably 4 and more preferably 5; the same applies hereinafter), more preferably 2 to 18, still more preferably 2 to 15, still more preferably 2 to 12, still more preferably 2 to 10, still more preferably 2 to 8, still more preferably 2 to 6, and still more preferably 2 to 5. The alkenyl group which can be adopted as R¹ is preferably an unsubstituted alkenyl group. The alkenyl group which can be adopted as R¹ is more preferably vinyl, allyl, or dimethylallyl.

The alkynyl group which can be adopted as R¹ may be linear or branched, or may form a ring. The number of carbon atoms in the alkynyl group is preferably 2 to 20 (in a case where the alkynyl group has a ring structure (cycloalkynyl group), the lower limit of the number of carbon atoms is 3, preferably 4 and more preferably 5; the same applies hereinafter), more preferably 2 to 18, still more preferably 2 to 15, still more preferably 2 to 12, still more preferably 2 to 10, still more preferably 2 to 8, still more preferably 2 to 6, and still more preferably 2 to 5. The alkynyl group which can be adopted as R¹ is preferably an unsubstituted alkynyl group. The alkynyl group that can be adopted as R¹ is more preferably ethynyl or propynyl.

Among these, R¹ is preferably a hydrogen atom or methyl.

In General Formula (1), R² represents a hydrogen atom, a chain-like aliphatic group, an aliphatic hydrocarbon ring group, an aryl group, or a heterocyclic group.

The chain-like aliphatic group which can be adopted as R² may be a saturated chain-like aliphatic group or an unsaturated chain-like aliphatic group. The number of carbon atoms in the chain-like aliphatic group which can be adopted as R² is preferably 1 to 20, more preferably 1 to 18, still more preferably 1 to 15, still more preferably 1 to 12, still more preferably 1 to 10, still more preferably 1 to 8, still more preferably 1 to 6, and still more preferably 1 to 5. The chain-like aliphatic group which can be adopted as R² is preferably a chain-like aliphatic hydrocarbon group. The chain-like aliphatic hydrocarbon group is more preferably an alkyl group, an alkenyl group, or an alkynyl group. Preferred aspects of the alkyl group, the alkenyl group, and the alkynyl group, which can be adopted as R², are respectively the same as the preferred aspects of the alkyl group, the alkenyl group, and the alkynyl group, which can be adopted as R¹ described above.

The aliphatic hydrocarbon ring group which can be adopted as R² may be a saturated aliphatic hydrocarbon ring group or an unsaturated aliphatic hydrocarbon ring group. In addition, a fused ring may be employed. The number of carbon atoms in the aliphatic hydrocarbon ring group which can be adopted as R² is preferably 3 to 20, more preferably 4 to 18, still more preferably 5 to 15, still more preferably 6 to 12, and still more preferably 6 to 10. The saturated aliphatic hydrocarbon ring group which can be adopted as R² is preferably a cycloalkyl group. In addition, the unsaturated aliphatic hydrocarbon ring group which can be adopted as R² is preferably a cycloalkenyl group or a cycloalkynyl group. The number of ring-constituting carbon atoms in the cycloalkyl group, the cycloalkenyl group, and the cycloalkynyl group which can be adopted as R² is preferably 4 to 10 and more preferably 5 to 8.

The number of carbon atoms in the aryl group which can be adopted as R² is preferably 6 to 40, more preferably 6 to 30, still more preferably 6 to 20, still more preferably 6 to 15, and still more preferably 6 to 12. The aryl group which can be adopted as R² is more preferably phenyl or naphthyl, and particularly preferably phenyl.

The number of ring-constituting atoms in the heterocyclic group which can be adopted as R² is preferably 3 to 20, more preferably 4 to 15, and more preferably 5 to 10. The heterocyclic ring may be aliphatic or aromatic. In addition, the heterocyclic group may have a fused ring structure. In a case where the heterocyclic group which can be adopted as R² is a monocycle, the number of ring-constituting atoms is preferably 5 or 6. Examples of the ring-constituting heteroatom (atom other than carbon atom) of the heterocyclic ring include boron (B), nitrogen (N), oxygen (O), sulfur (S), selenium (Se), and tellurium (Te), and it is preferable to have a heteroatom selected from nitrogen, oxygen, and sulfur. Preferred specific examples of the heterocyclic ring constituting the heterocyclic group which can be adopted as R² include, as a saturated heterocyclic ring, a pyrrolidine ring, an imidazolidine ring, a pyrazolidine ring, a piperidine ring, a piperazine ring, a morpholine ring, a 2-bora-1,3-dioxolane ring, and a 1,3-thiazolidine ring. In addition, examples thereof include, as an unsaturated heterocyclic ring, a pyrrole ring, an imidazole ring, a thiophene ring, a pyrazole ring, an oxazole ring, an isoxazole ring, a thiazole ring, a triazole ring, a tetrazole ring, a furan ring, a benzothiazole ring, a benzoxazole ring, a benzotriazole ring, a benzoselenazole ring, a benzofuran ring, a benzothiophene ring, a pyridine ring, a pyrazine ring, a pyrimidine ring, a pyridazine ring, a triazine ring, a quinoline ring, an isoquinoline ring, a quinoxaline ring, a 2-pyrroline ring, a 2-imidazoline ring, and a 3-pyrazoline ring.

Preferred specific examples of the compound represented by General Formula (1) include acrylic acid, methacrylic acid, methyl acrylate, methyl methacrylate, ethyl acrylate, ethyl methacrylate, phenyl acrylate, phenyl methacrylate, butyl acrylate, butyl methacrylate, isobutyl acrylate, isobutyl methacrylate, vinyl acrylate, vinyl methacrylate, allyl acrylate, allyl methacrylate, isopropyl acrylate, isopropyl methacrylate, tert-butyl acrylate, tert-butyl methacrylate, dodecyl acrylate, dodecyl methacrylate, hexyl acrylate, hexyl methacrylate, stearyl acrylate, stearyl methacrylate, 2-(chloromethyl)ethyl acrylate, 2-(chloromethyl)methyl acrylate, 2-(chloromethyl)acrylic acid, itaconic acid, diethyl itaconate, β-methyl itaconate, dimethyl itaconate, and tert-butyl 2-(trifluoromethyl)acrylate.

In General Formula (2), a ring Ar represents an aromatic ring.

In a case where the aromatic ring which can be adopted as the ring Ar is an aromatic hydrocarbon ring, the number of carbon atoms in the aromatic hydrocarbon ring is preferably 6 to 40, more preferably 6 to 30, still more preferably 6 to 20, still more preferably 6 to 15, and still more preferably 6 to 12. The aromatic hydrocarbon ring which can be adopted as the ring Ar may be a monocyclic ring or a fused ring. Preferred specific examples of the aromatic hydrocarbon ring include a benzene ring and a naphthalene ring, and among these, a benzene ring is preferable.

In a case where the aromatic ring which can be adopted as the ring Ar is an aromatic heterocyclic ring, the aromatic heterocyclic ring may be a monocyclic ring or a fused ring. The number of ring-constituting atoms in the aromatic heterocyclic ring is preferably 5 to 20, more preferably 5 to 15, and still more preferably 5 to 10. In addition, in a case where the aromatic heterocyclic ring is a monocyclic ring, the number of ring-constituting atoms is preferably 5 or 6. Examples of the ring-constituting heteroatom (atom other than carbon atom) of the aromatic heterocyclic ring include boron (B), nitrogen (N), oxygen (O), sulfur (S), and selenium (Se), and it is preferable to have a heteroatom selected from nitrogen, oxygen, and sulfur. Specific examples of the aromatic heterocyclic ring which can be adopted as the ring Ar include a pyrrole ring, an imidazole ring, a thiophene ring, a pyrazole ring, an oxazole ring, an isoxazole ring, a thiazole ring, a triazole ring, a tetrazole ring, a furan ring, a benzothiazole ring, a benzoxazole ring, a benzotriazole ring, a benzoselenazole ring, a benzofuran ring, a benzothiophene ring, a pyridine ring, a pyrazine ring, a pyrimidine ring, a pyridazine ring, a triazine ring, a quinoline ring, an isoquinoline ring, and a quinoxaline ring.

R³ represents an electron withdrawing group. The electron withdrawing group usually refers to a substituent having a positive Hammett's σ value. The Hammett's rule is an empirical rule advocated by L. P. Hammett in 1935 so as to quantitatively discuss the effect of substituent on the reaction or equilibrium of benzene derivatives and its propriety is widely admitted at present. A substituent constant according to the Hammett's rule can be found in general textbooks, and for example, “Lange's Handbook of Chemistry” 12th Edition, edited by J. A. Dean, 1979 (Mc Graw-Hill) and Special Issue of “Field of Chemistry”, No. 122, pages 96 to 103, 1979 (Nankodo Co., Ltd.) can be referred to.

Examples of the electron withdrawing group which can be adopted as R³ include an acyl group (preferably having 2 to 20 carbon atoms, more preferably having 2 to 10 carbon atoms, and still more preferably having 2 to 5 carbon atoms), an alkoxycarbonyl group (preferably having 2 to 20 carbon atoms, more preferably having 2 to 10 carbon atoms, and still more preferably having 2 to 5 carbon atoms), an aryloxycarbonyl group (preferably having 7 to 20 carbon atoms and more preferably having 7 to 10 carbon atoms), a carbamoyl group, an alkylsulfonyl group (preferably having 1 to 20 carbon atoms, more preferably having 1 to 10 carbon atoms, and still more preferably having 2 to 5 carbon atoms), an arylsulfonyl group (preferably having 6 to 20 carbon atoms and more preferably having 6 to 10 carbon atoms), a sulfamoyl group, a trifluoromethyl group, a cyano group, a nitro group, and a halogen atom (for example, a fluorine atom and a chlorine atom). A group selected from an acyl group, an alkoxycarbonyl group, an aryloxycarbonyl group, a carbamoyl group, an alkylsulfonyl group, an arylsulfonyl group, a sulfamoyl group, a cyano group, a nitro group, or a halogen atom is preferable; a group selected from an acyl group, an alkoxycarbonyl group, an aryloxycarbonyl group, a carbamoyl group, a sulfamoyl group, a cyano group, or a halogen atom is more preferable; and a group selected from an acyl group, a sulfamoyl group, or a halogen atom is still more preferable.

m representing the number of R³'s is an integer of 1 or more. That is, in the compound represented by General Formula (2), the ring Ar has one or two or more electron withdrawing groups as a substituent. In a case where the ring Ar has two or more electron withdrawing groups R³, the two or more electron withdrawing groups R³ may be the same or different from each other.

R⁴ represents a chain-like aliphatic group, an aliphatic hydrocarbon ring group, an aryl group, or a heterocyclic group.

The chain-like aliphatic group, the aliphatic hydrocarbon ring group, the aryl group, and the heterocyclic group, which can be adopted as R⁴, are respectively the same as the chain-like aliphatic group, the aliphatic hydrocarbon ring group, the aryl group, and the heterocyclic group, which can be adopted as R² described above, and preferred aspects thereof are also the same. However, R⁴ is not an α-hydroxybenzyl group. In a case where R⁴ is an α-hydroxybenzyl group, the side reaction is especially likely to proceed, which gives many by-products. From the same viewpoint, it is more preferable that R⁴ does not have a hydroxy group. Furthermore, it is preferable that the compound represented by General Formula (2) is a compound not having a hydroxy group as a substituent.

n representing the number of R⁴'s is an integer of 0 or more. In a case where the ring Ar has two or more R⁴'s, the two or more R⁴'s may be the same or different from each other.

The maximum value (upper limit) of the total number of m and n (m+n) is the maximum value of the number of substituents which can be included in the ring-constituting atoms of the ring Ar. For example, in a case where the ring Ar in General Formula (2) is a benzene ring, since the ring Ar already has an amino group (—NH₂) as a substituent, the maximum value of substituents which can be included in the ring-constituting atoms of the ring Ar is 5.

In General Formula (2), it is preferable that m is an integer of 1 to 3 (preferably 1 or 2 and more preferably 1) and n is an integer of 0 to 4 (preferably an integer of 0 to 3, more preferably an integer of 0 to 2, and still more preferably 0 or 1). In this case, the ring Ar is preferably a 5-membered ring or a 6-membered ring, and more preferably a benzene ring.

Preferred specific examples of the compound represented by General Formula (2) include sulfanylamide, 4-fluoroaniline, 4-aminoacetophenone, 2,4-difluoroaniline, 4-chloroaniline, 2-methyl-4-fluoroaniline, 4-bromoaniline, 2,4-dibromoaniline, 2,4-dichloroaniline, 2,4,6-trifluoroaniline, 2-fluoroaniline, pentafluoroaniline, 3-chloro-4-fluoroaniline, 4-trifluoromethylaniline, 4-nitroaniline, 2-fluoro-5-methylaniline, 4-aminobenzophenone, 2′-aminoacetophenone, 4′-amino-3′,5′-dichloroacetophenone, 2-trifluoromethylaniline, 2-iodo-4-(trifluoromethyl)aniline, 4-amino-3-chlorobenzotrifluoride, 4-amino-3-bromobenzotrifluoride, 4-amino-3,5-dichlorobenzotrifluoride, 2-nitroaniline, 1-amino-4-fluoronaphthalene, 1-amino-4-bromonaphthalene, 1-amino-4-chloronaphthalene, 1-amino-4-nitronaphthalene, and 3-amino-4-(trifluoromethyl)pyridine.

R¹, Ar, R³, R⁴, m, and n in General Formula (3) are respectively the same as R¹, Ar, R³, R⁴, m, and n described in General Formula (1) or General Formula (2), and preferred aspects thereof are also the same.

<Substituent Group Z>

Halogen atom (for example, a fluorine atom, a chlorine atom, a bromine atom, or an iodine atom); an alkyl group [representing a linear, branched, or cyclic substituted or unsubstituted alkyl group; the alkyl group includes an alkyl group (preferably an alkyl group having 1 to 30 carbon atoms; for example, methyl, ethyl, n-propyl, isopropyl, t-butyl, n-octyl, eicosyl, 2-chloroethyl, 2-cyanoethyl, and 2-ethylhexyl), a cycloalkyl group (preferably a substituted or unsubstituted cycloalkyl group having 3 to 30 carbon atoms; for example, cyclohexyl, cyclopentyl, and 4-n-dodecylcyclohexyl), a bicycloalkyl group (preferably a substituted or unsubstituted bicycloalkyl group having 5 to 30 carbon atoms, that is, a monovalent group obtained by removing one hydrogen atom from a bicycloalkane having 5 to 30 carbon atoms; for example, bicyclo[1,2,2]heptan-2-yl and bicyclo[2,2,2]octan-3-yl), a tricyclo structure having many ring structures, and the like; an alkyl group in the substituent described below (for example, an alkyl group in the alkylthio group) also represents an alkyl group of this concept];

an alkenyl group [representing a linear, branched, or cyclic substituted or unsubstituted alkynyl group; the alkenyl group includes an alkenyl group (preferably a substituted or unsubstituted alkenyl group having 2 to 30 carbon atoms; for example, vinyl, allyl, prenyl, geranyl, and oleyl), a cycloalkenyl group (preferably a substituted or unsubstituted cycloalkenyl group having 3 to 30 carbon atoms, that is, a monovalent group obtained by removing one hydrogen atom from a cycloalkene having 3 to 30 carbon atoms; for example, 2-cyclopenten-1-yl and 2-cyclohexen- 1-yl), and a bicycloalkenyl group (a substituted or unsubstituted bicycloalkenyl group, preferably a substituted or unsubstituted bicycloalkenyl group having 5 to 30 carbon atoms, that is, a monovalent group obtained by removing one hydrogen atom from a bicycloalkene with one double bond; for example, bicyclo[2,2,1]hept-2-en-1-yl and bicyclo[2,2,2]oct-2-en-4-yl); an alkynyl group (preferably a substituted or unsubstituted alkynyl group having 2 to 30 carbon atoms; for example, ethynyl, propargyl, and a trimethylsilylethynyl group);

an aryl group (preferably a substituted or unsubstituted aryl group having 6 to 30 carbon atoms; for example, phenyl, p-tolyl, naphthyl, m-chlorophenyl, and o-hexadecanoylaminophenyl), a heterocyclic group (preferably a substituted or unsubstituted 5- or 6-membered monovalent group obtained by removing one hydrogen atom from an aromatic or non-aromatic heterocyclic compound, and more preferably a 5- or 6-membered aromatic heterocyclic group having 3 to 30 carbon atoms; for example, 2-furyl, 2-thienyl, 2-pyrimidinyl, and 2-benzothiazolyl), a cyano group, a nitro group, a carboxyl group, an alkoxy group (preferably a substituted or unsubstituted alkoxy group having 1 to 30 carbon atoms; for example, methoxy, ethoxy, isopropoxy, t-butoxy, n-octyloxy, and 2-methoxyethoxy); an aryloxy group (preferably a substituted or unsubstituted aryloxy group having 6 to 30 carbon atoms; for example, phenoxy, 2-methylphenoxy, 4-t-butylphenoxy, 3-nitrophenoxy, and 2-tetradecanoylaminophenoxy);

a silyloxy group (preferably a silyloxy group having 3 to 20 carbon atoms; for example, trimethylsilyloxy and t-butyldimethylsilyloxy), a heterocyclic oxy group (preferably a substituted or unsubstituted heterocyclic oxy group having 2 to 30 carbon atoms; 1-phenyltetrazol-5-oxy and 2-tetrahydropyranyloxy), an acyloxy group (preferably a formyloxy group, a substituted or unsubstituted alkylcarbonyloxy group having 2 to 30 carbon atoms, or a substituted or unsubstituted arylcarbonyloxy group having 6 to 30 carbon atoms; for example, formyloxy, acetyloxy, pivaloyloxy, stearoyloxy, benzoyloxy, and p-methoxyphenylcarbonyloxy); a carbamoyloxy group (preferably a substituted or unsubstituted carbamoyloxy group having 1 to 30 carbon atoms; for example, N,N-dimethylcarbamoyloxy, N,N-diethylcarbamoyloxy, morpholinocarbonyloxy, N,N-di-n-octylaminocarbonyloxy, and N-n-octylcarbamoyloxy), an acyl group (preferably a formyl group, a substituted or unsubstituted alkylcarbonyl group having 2 to 30 carbon atoms, a substituted or unsubstituted arylcarbonyl group having 7 to 30 carbon atoms, or a heterocyclic carbonyl group bonded to a carbonyl group at a substituted or unsubstituted carbon atom, having 4 to 30 carbon atoms; for example, acetyl, pivaloyl, 2-chloroacetyl, stearoyl, benzoyl, p-n-octyloxyphenylcarbonyl, 2-pyridylcarbonyl, and 2-furylcarbonyl); an aryloxycarbonyl group (preferably a substituted or unsubstituted aryloxycarbonyl group having 7 to 30 carbon atoms; for example, phenoxycarbonyl, o-chlorophenoxycarbonyl, m-nitrophenoxycarbonyl, and p-t-butylphenoxycarbonyl); an alkoxycarbonyl group (preferably a substituted or unsubstituted alkoxycarbonyl group having 2 to 30 carbon atoms; for example, methoxycarbonyl, ethoxycarbonyl, t-butoxycarbonyl, and n-octadecyloxycarbonyl); a carbamoyl group (preferably a substituted or unsubstituted carbamoyl having 1 to 30 carbon atoms; for example, carbamoyl, N-methylcarbamoyl, N,N-dimethylcarbamoyl, N,N-di-n-octylcarbamoyl, and N-(methylsulfonyl)carbamoyl);

an aryl or heterocyclic azo group (preferably a substituted or unsubstituted aryl azo group having 6 to 30 carbon atoms or a substituted or unsubstituted heterocyclic azo group having 3 to 30 carbon atoms; for example, phenylazo, p-chlorophenylazo, and 5-ethylthio-1,3,4-thiadiazol-2-ylazo); an imide group (preferably N-succinimide or N-phthalimide); a phosphino group (preferably a substituted or unsubstituted phosphino group having 2 to 30 carbon atoms; for example, dimethylphosphino, diphenylphosphino, and methylphenoxyphosphino); a phosphinyl group (preferably a substituted or unsubstituted phosphinyl group having 2 to 30 carbon atoms; for example, phosphinyl, dioctyloxyphosphinyl, and diethoxyphosphinyl); a phosphinyloxy group (preferably a substituted or unsubstituted phosphinyloxy group having 2 to 30 carbon atoms; for example, diphenoxyphosphinyloxy and dioctyloxyphosphinyloxy); a phosphinylamino group (preferably a substituted or unsubstituted phosphinylamino group having 2 to 30 carbon atoms; for example, dimethoxyphosphinylamino and dimethylaminophosphinylamino); and a silyl group (preferably a substituted or unsubstituted silyl group having 3 to 30 carbon atoms; for example, trimethylsilyl, t-butyldimethylsilyl, and phenyldimethylsilyl).

Among the above-described functional groups, those having a hydrogen atom may be removed and further substituted with a group selected from the substituent group Z.

The production method according to the embodiment of the present invention is characterized in that the above-described compound represented by General Formula (1) is reacted with the above-described compound represented by General Formula (2) at a temperature of higher than 120° C. to carry out amidation. By carrying out the amidation reaction at a high temperature of higher than 120° C., it is possible to effectively suppress the formation of the 1,4-adduct which is a by-product, and it is possible to obtain the compound represented by General Formula (3), which is the target 1,2-adduct, with high efficiency.

Here, in a case where a compound having no electron withdrawing group R³ in General Formula (2) is used as a raw material amine compound, the problem of regioselectivity of the reaction is not actualized. That is, the target 1,2-adduct can be efficiently obtained to a certain extent even in a case where the temperature is not controlled to a high temperature. On the other hand, according to the studies by the present inventors, it has been found that, in the compound represented by General Formula (2), which is the raw material amine compound, the ring Ar has an electron withdrawing group, and the by-product 1,4-adduct is likely to be produced. For example, in a case where sulfanylamide having a sulfamoyl group as the electron withdrawing group R³ is used as the compound represented by General Formula (2), as shown in Comparative Examples described later, it has been found that, even in a case where the reaction is carried out at a high temperature of approximately 100° C., the amount of 1,4-adduct produced is approximately 10 times or more in terms of molar ratio with respect to the desired amount of 1,2-adduct produced. The present invention has been made in order to deal with this novel problem.

In the production method according to the embodiment of the present invention, the reaction temperature is controlled to be in a high temperature range of higher than 120° C. By controlling the reaction temperature, it is possible to rapidly increase efficiency of producing the target 1,2-adduct without using an expensive raw material activated form or without using a reagent such as a condensing agent. The reason is not clear, but is presumed as follows.

In a case where the compound represented by General Formula (1) is reacted with the compound represented by General Formula (2), it is considered that the following two reactions mainly occur. In the following scheme, a case where methacrylic acid is applied as the compound represented by General Formula (1) and sulfanylamide is applied as the compound represented by General Formula (2) is shown.

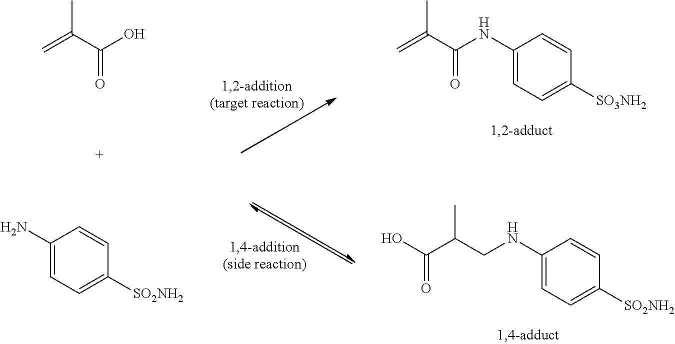

The 1,4-addition reaction is reversible, and it is considered that a reverse reaction (retro-Michael reaction) of the 1,4-addition reaction is promoted by carrying out the reaction in a specific high temperature range, converging on the formation of the 1,2-adduct. Until now, the temperature range at which the retro-Michael reaction occurs has not been known. In addition, in a situation where it is known that side reactions are likely to occur in a case where the reaction is at a high temperature, at the time of filing the application, it has been completely unknown how the high-temperature reaction defined in the present invention affects the amidation reaction. According to the present invention, by controlling the reaction temperature of the amidation reaction within a specific high temperature range, it is possible to remarkably improve production efficiency of the target 1,2-adduct without requiring any other special device and using inexpensive raw materials.

In the reaction between the compound represented by General Formula (1) and the compound represented by General Formula (2) (amidation reaction) of the production method according to the embodiment of the present invention, the reaction temperature may be controlled to a temperature of higher than 120° C., a batch-type reaction may be used or a flow-type (circulation-type) reaction may be used in which the reaction is carried out while a raw material mixed solution (meaning a reaction solution before the start of the reaction, and in a case of using a solvent, a catalyst, an additive, or the like in addition to the raw materials, meaning a mixed solution containing these substances) is circulated in a flow channel. The flow-type reaction itself is well-known, and for example, WO2020/066561A, WO2019/188749A, WO2018/180456A, JP2016-160124A, and the like can be appropriately referred to.

A method of controlling the reaction temperature to higher than 120° C. is not particularly limited, and for example, the reaction temperature can be controlled using a constant-temperature tank. In addition, it is also preferable to control the temperature by heating the raw material mixed solution with microwave irradiation. By applying microwave heating, the raw material mixed solution can be instantly heated to a target high temperature in a non-contact manner, and reaction conditions of the amidation reaction can be accurately controlled. In the production method according to the embodiment of the present invention, it is also preferable to carry out the amidation reaction in a flow -type reaction and to control the temperature in the flow-type reaction by microwave irradiation.

The reaction temperature of the above-described amidation reaction is preferably 121° C. or higher, more preferably 122° C. or higher, still more preferably 123° C. or higher, still more preferably 124° C. or higher, and still more preferably 125° C. or higher. In addition, the above-described reaction temperature is also preferably 130° C. or higher, preferably 140° C. or higher, preferably 150° C. or higher, preferably 160° C. or higher, preferably 180° C. or higher, preferably 200° C. or higher, preferably 205° C. or higher, preferably 210° C. or higher, and preferably 220° C. or higher. As the temperature is higher, the retro-Michael reaction can be promoted more. In addition, from the viewpoint of preventing excessive pressure rise in the reaction system, the reaction temperature of the above-described amidation reaction is generally 500° C. or lower, preferably 400° C. or lower, preferably 350° C. or lower, preferably 300° C. or lower, or preferably 280° C. or lower.

In the production method according to the embodiment of the present invention, in a case where the amidation reaction is carried out the batch type, the raw material mixed solution is usually subjected to a heating treatment after being sufficiently stirred. In addition, it is also preferable to subject the raw material mixed solution to a heating treatment while stirring the raw material mixed solution.

In a case where the above-described amidation reaction is carried out by the flow-type reaction, the raw material mixed solution is heated while being allowed to flow into the flow channel to cause the amidation reaction. The flow-type reaction has an advantage that a reaction product can be continuously obtained while continuously supplying raw materials. For example, the amidation reaction can be carried out by mixing the raw material mixed solution in a container, introducing this mixed solution into a flow channel, and heating the mixed solution while flowing downstream. In addition, the amidation reaction can also be carried out by respectively flowing a solution containing the compound represented by General Formula (1) and a solution containing the compound represented by General Formula (2) in different flow channels, joining these flow channels, and heating the joined solutions while flowing downstream.

In the above-described amidation reaction, amounts of the compound represented by General Formula (1) and the compound represented by General Formula (2) used are not particularly limited as long as the compound represented by General Formula (3), which is the target 1,2-adduct, can be obtained. In a case where the amount of the compound represented by General Formula (2) used is large, the amount of the by-product 1,4-adduct produced tends to increase, so that the compound represented by General Formula (1) is generally reacted in an excess amount on a molar basis over the compound represented by General Formula (2). For example,
it is preferable that 1.2≤[Molar amount of compound represented by General Formula (1)]/[Molar amount of compound represented by General Formula (2)],
it is more preferable that 1.5≤[Molar amount of compound represented by General Formula (1)]/[Molar amount of compound represented by General Formula (2)],
it is still more preferable that 1.8≤[Molar amount of compound represented by General Formula (1)]/[Molar amount of compound represented by General Formula (2)], and
it is still more preferable that 2.0≤[Molar amount of compound represented by General Formula (1)]/[Molar amount of compound represented by General Formula (2)].

In addition, from the viewpoint of obtained amount,
it is preferable that [Molar amount of compound represented by General Formula (1)]/[Molar amount of compound represented by General Formula (2)]≤10.0,
it is more preferable that [Molar amount of compound represented by General Formula (1)]/[Molar amount of compound represented by General Formula (2)]≤8.0, and
it is still more preferable that [Molar amount of compound represented by General Formula (1)]/[Molar amount of compound represented by General Formula (2)]≤7.0.

It is also preferable to use a solvent for the above-described amidation reaction. By using a solvent, viscosity of the raw material mixed solution can be lowered, and it is considered that the generation of side reactions can be suppressed more effectively as mixing efficiency is improved. As the solvent, an organic solvent in which the reaction raw materials can be dissolved is usually used. From the viewpoint of suppressing pressure increase, the above-described solvent is preferably a solvent having a boiling point of 100° C. or higher, and more preferably a solvent having a boiling point of 150° C. or higher. This boiling point is a boiling point at 0.1 MPa. In a case where an alcohol-based solvent, an ester-based solvent, or an acyclic amide-based solvent not having a urea bond is used as the above-described solvent, the progress of the above-described amidation reaction may be hindered, so that it is preferable to use a solvent other than these solvents. Examples of a preferred solvent include a nitrile -based solvent (solvent including a compound having a nitrile group), an ether-based solvent (solvent including a compound having an ether bond), an aliphatic hydrocarbon-based solvent (solvent including an aliphatic hydrocarbon compound), an aromatic hydrocarbon-based solvent (solvent including an aromatic hydrocarbon compound), a carbonate-based solvent (solvent including a carbonate ester compound), a ketone-based solvent (solvent including a ketone compound), a sulfoxide-based solvent (solvent including a sulfoxide compound), a sulfone-based solvent (solvent including a sulfone compound), a cyclic amide-based solvent (solvent including a cyclic amide compound), and a urea-based solvent (solvent including a compound having a urea bond).

Examples of the nitrile-based solvent include acetonitrile and propionitrile.

Examples of the ether-based solvent include diethyl ether, dibutyl ether, diisopropyl ether, t-butyl methyl ether, cyclopentyl methyl ether, tetrahydrofuran, 2-methyltetrahydrofuran, 4-methyltetrahydropyran, and 1,4-dioxane.

Examples of the aliphatic hydrocarbon-based solvent include hexane, heptane, octane, and decane.

Examples of the aromatic hydrocarbon-based solvent include benzene, toluene, xylene, dichlorobenzene, benzotrifluoride, and nitrobenzene.

Examples of the carbonate-based solvent include ethylene carbonate and propylene carbonate.

Examples of the sulfoxide-based solvent include dimethyl sulfoxide.

Examples of the sulfone-based solvent include 3-methylsulfolane and sulfolane.

Examples of the cyclic amide-based solvent include N-methyl-2-pyrrolidone.

Examples of the urea-based solvent include 1,3-dimethyl-2-imidazolidinone, N,N′-dimethylpropylene urea, and N,N,N′,N′-tetramethylurea.

In a case where the solvent is used in the above-described amidation reaction, the amount of the solvent used can be appropriately set in consideration of the viscosity of the raw material mixed solution, the consideration of the reaction product, and the like. For example, the amount of the solvent used can be 1 to 100 parts by mass with respect to the total amount of 100 parts by mass of the compound represented by General Formula (1) and the compound represented by General Formula (2), preferably 5 to 60 parts by mass and preferably 10 to 30 parts by mass.

It is also preferable to use a catalyst for the above-described amidation reaction. By using a catalyst, it is possible to further enhance the regioselectivity of the reaction. It is preferable to use at least one of a Lewis acid, a Broensted acid, a metal oxide, or a phosphorus oxide compound as a reaction catalyst.

The Lewis acid is a substance which can receive an electron pair. Examples of the Lewis acid catalyst which can be used in the above-described amidation reaction include BF₃·OEt₂, AlBr₃, AlCl₃, ZnI₂, MgCl₂, TiCl₄, TiCl₃(OiPr), TiCl₂(OiPr)₂, TiCl(OiPr)₃, Ti(OiPr)₄, SnCl₄, SnCl₃, EtAlCl₂, FeCl₃, ZnCl₂, TMSOTf, FeBr₃, BBr₃, Sc(OTf)₂, Zn(OTf)₂, La(OTf)₃, Yb(OTf)₃, Hf(OTf)₄, BeCl₂, CdCl₂, GaCl₃, and SbCl₅. Among these, a titanium compound is preferable, and TiCl₄ is more preferable. In the formulae, Et represents ethyl, iPr represents isopropyl, Tf represents trifluoromethylsulfonyl, and TMS represents trimethylsilyl.

In the present invention, in a case where the Lewis acid is used as a reaction catalyst, one kind or two or more kinds of the above-described Lewis acid can be used.

The Broensted acid is an acid which has a proton and can release or dissociate the proton. Specific examples of the Broensted acid catalyst include hydrochloric acid, nitric acid, sulfuric acid, hydrofluoric acid, boric acid, formic acid, acetic acid, trichloroacetic acid, trifluoroacetic acid, methanesulfonic acid, benzenesulfonic acid, p-toluenesulfonic acid, 10-camphorsulfonic acid, and Amberlyst (registered trademark) 15 hydrogen form. Among these, a sulfonic acid compound is preferable.

In the present invention, in a case where the Broensted acid is used as a reaction catalyst, one kind or two or more kinds of the above-described Broensted acid can be used.

The metal oxide is not particularly limited as long as it is an oxide of a metal. Examples thereof include SiO₂, SiO, MgO, Al₂O₃, GeO, NiO, SrO, Y₂O₃, ZrO₂, CeO₂, Fe₂O₃, Rb₂O, Sc₂O₃, La₂O₃, Nd₂O₃, Sm₂O₃, Gd₂O₃, Dy₂O₃, Er₂O₃, Yb₂O₃, Ta₂O₃, Ta₂O₅, Nb₂O₅, HfO₂, Ga₂O₃, and TiO₂. In addition, a mixture including a metal oxide such as zeolite and clay mineral can also be used. Among these, TiO₂ is preferable.

In the present invention, in a case where the metal oxide is used as a reaction catalyst, one kind or two or more kinds of the above-described metal oxides can be used.

The phosphorus oxide compound is a compound having an oxygen atom directly bonded to a phosphorus atom. In a case where the phosphorus oxide compound is an acid capable of releasing or dissociating protons, such as phosphoric acid and polyphosphoric acid, the phosphorus oxide compound is the Broensted acid. However, in the present invention or the specification, for convenience, a compound which is both the Broensted acid and the phosphorus oxide compound is positioned as the phosphorus oxide compound, not the Broensted acid described above. Specific examples of the phosphorus oxide compound include diphosphorus pentoxide, hypophosphorous acid, phosphorous acid, and phosphoric acid. In addition, polymerized phosphoric acids (polyphosphoric acids) such as pyrrolic acid, triphosphoric acid, trimetaphosphoric acid, and tetrametaphosphoric acid are also preferable as the phosphorus oxide compound. Among these, diphosphorus pentoxide has an action of suppressing elimination of the electron withdrawing group in the reaction at a high temperature, which is preferable. It is presumed that this is because the diphosphorus pentoxide traps water which is a starting point of the above-described elimination reaction. An Eaton reagent can also be used as the diphosphorus pentoxide.

In the present invention, in a case where the phosphorus oxide compound is used as a reaction catalyst, one kind or two or more kinds of the above-described phosphorus oxide compounds can be used.

A reaction time (time of exposure to a temperature of higher than 120° C.) of the above-described amidation reaction is not particularly limited, and it is appropriately adjusted within a range in which a sufficient amount of the target reaction product can be obtained. For example, the reaction time can be set to 1 to 300 minutes, preferably set to 2 to 240 minutes, preferably set to 3 to 120 minutes, and preferably set to 4 to 90 minutes. In a case where precise temperature control such as microwave heating is performed, the reaction time can be further shortened. The reaction can be terminated by, for example, cooling.

In the production method according to the embodiment of the present invention, in order to prevent unsaturated double bond of the compound represented by General Formula (1) from causing an addition polymerization reaction, it is also preferable to add a polymerization inhibitor. A general polymerization inhibitor can be used, and for example, TEMPO, 4-hydroxy TEMPO, or the like can be appropriately used.

In the production method according to the embodiment of the present invention, the target compound represented by General Formula (3) can be produced in a reaction solution as a main reaction product by the amidation reaction. In the reaction solution after the amidation reaction (unpurified reaction solution), an amount ratio of the compound represented by General Formula (3) (1,2-adduct) to the by-product 1,4-adduct is, in terms of molar ratio,
preferably 0.3≤[1,2-adduct]/[1,4-adduct],
more preferably 0.5≤[1,2-adduct]/[1,4-adduct],
more preferably 0.8≤[1,2-adduct]/[1,4-adduct],
more preferably 1.1≤[1,2-adduct]/[1,4-adduct],
more preferably 1.2≤[1,2-adduct]/[1,4-adduct],
more preferably 1.4 <[1,2-adduct]/[1,4-adduct],
more preferably 1.7 <[1,2-adduct]/[1,4-adduct], and
still more preferably 2.0 <[1,2-adduct]/[1,4-adduct].

The upper limit of the above-described molar ratio is not limited, and is generally
[1,2-adduct]/[1,4-adduct]≤10.0,
and may be [1,2-adduct]/[1,4-adduct]≤8.0,
[1,2-adduct]/[1,4-adduct]≤6.0,
[1,2-adduct]/[1,4-adduct]≤5.0, or
[1,2-adduct]/[1,4-adduct]≤4.0.

It is also possible to separate and purify the compound represented by General Formula (3), which is the target 1,2-adduct, from the reaction solution after the amidation reaction. As this separation or purification method, a general method can be appropriately applied. For example, flash column chromatography, thin layer column chromatography, crystallization, recrystallization, distillation, or the like can be applied alone or in combination.

Hereinafter, the present invention will be more specifically described based on Examples, but the present invention is not limited to Examples.

### EXAMPLES

### Example 1

10 mg of 4-hydroxy TEMPO, 1.0 g (5.8 mmol, 1.0 eq.) of sulfanylamide, and 1.25 g (14.5 mmol, 2.5 eq.) of methacrylic acid were charged into a 2 mL vial for microwave reaction to prepare a reaction mixed solution, and the vial was sealed. The vial was set in a microwave reactor manufactured by Biotage Ltd., and an amidation reaction was carried out by setting reaction conditions as follows. No solvent was used in the reaction system.

<Reaction Conditions>
Set temperature: 250° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.4/1.0 (molar ratio).

### Example 2

5 mg of 4-hydroxy TEMPO, 0.5 g (2.9 mmol, 1.0 eq.) of sulfanylamide, 0.62 g (7.3 mmol, 2.5 eq.) of methacrylic acid, and 0.62 mL of sulfolane as a solvent were charged and mixed in a 2 mL vial for microwave reaction to prepare a reaction mixed solution, and the vial was sealed. The vial was set in a microwave reactor manufactured by Biotage Ltd., and an amidation reaction was carried out by setting reaction conditions as follows.
Set temperature: 250° C.
Pre-stirring: 2 minutes
Reaction time: 20 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.4/1.0 (molar ratio).

### Example 3

5 mg of 4-hydroxy TEMPO, 0.5 g (2.9 mmol, 1.0 eq.) of sulfanylamide, and 1.25 g (14.5 mmol, 5.0 eq.) of methacrylic acid were mixed in a 2 mL vial for microwave reaction, 55 mg (0.29 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 250° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=1.2/1.0 (molar ratio).

### Example 4

5 mg of 4-hydroxy TEMPO, 0.5 g (2.9 mmol, 1.0 eq.) of sulfanylamide, 0.62 g (7.3 mmol, 2.5 eq.) of methacrylic acid, and 0.62 mL of sulfolane as a solvent were mixed in a 2 mL vial for microwave reaction, 55 mg (0.29 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 250° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=3.1/1.0 (molar ratio).

### Example 5

5 mg of 4-hydroxy TEMPO, 0.5 g (2.9 mmol, 1.0 eq.) of sulfanylamide, 1.2 g (15 mmol, 5.0 eq.) of methacrylic acid, and 0.25 mL of 1,3-dimethyl-2-imidazolidinone (DMI) as a solvent were mixed in a 2 mL vial for microwave reaction, 55 mg (0.29 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 250° C.
Pre-stirring: 2 minutes
Reaction time: 10 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=3.4/1.0 (molar ratio).

2 mL of acetonitrile/water=1/2 (molar ratio) was added to the vial after the completion of the reaction, and the mixture was stirred at room temperature for 30 minutes. The obtained solid was collected by suction filtration, and washed with 1 mL of acetonitrile/water =1/2 (molar ratio). The obtained solid was dried under reduced pressure to obtain 0.21 g of a target 1,2-adduct at a yield of 30%.

### Example 6

5 mg of 4-hydroxy TEMPO, 0.5 g (2.9 mmol, 1.0 eq.) of sulfanylamide, 0.62 g (7.3 mmol, 2.5 eq.) of methacrylic acid, and 0.62 mL of sulfolane as a solvent were mixed in a 2 mL vial for microwave reaction, 55 mg (0.29 mmol, 0.1 eq.) of butyl acetate as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 150° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=1.2/1.0 (molar ratio).

### Example 7

5 mg of 4-hydroxy TEMPO, 0.5 g (3.7 mmol, 1.0 eq.) of 4′-aminoacetophenone, 1.6 g (18 mmol, 5.0 eq.) of methacrylic acid, and 0.25 mL of 1,3-dimethyl-2-imidazolidinone (DMI) as a solvent were mixed in a 2 mL vial for microwave reaction, 70 mg (0.37 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 150° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.1/1.0 (molar ratio).

### Example 8

5 mg of 4-hydroxy TEMPO, 0.5 g (4.0 mmol, 1.0 eq.) of 4-fluoro-2-methylaniline, 1.7 g (20 mmol, 5.0 eq.) of methacrylic acid, and 0.25 mL of 1,3-dimethyl-2-imidazolidinone (DMI) as a solvent were mixed in a 2 mL vial for microwave reaction, 76 mg (0.40 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 150° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.7/1.0 (molar ratio).

### Example 9

5 mg of 4-hydroxy TEMPO, 0.5 g (2.9 mmol, 1.0 eq.) of sulfanylamide, 1.5 g (15 mmol, 5.0 eq.) of methyl methacrylate, and 0.25 mL of 1,3-dimethyl-2-imidazolidinone (DMI) as a solvent were mixed in a 2 mL vial for microwave reaction, 55 mg (0.29 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 250° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.8/1.0 (molar ratio).

### Example 10

5 mg of 4-hydroxy TEMPO, 0.5 g (2.9 mmol, 1.0 eq.) of sulfanylamide, 2.4 g (15 mmol, 5.0 eq.) of phenyl methacrylate, and 0.25 mL of 1,3-dimethyl-2-imidazolidinone (DMI) as a solvent were mixed in a 2 mL vial for microwave reaction, 55 mg (0.29 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 250° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=1.9/1.0 (molar ratio).

### Example 11

5 mg of 4-hydroxy TEMPO, 0.5 g (3.7 mmol, 1.0 eq.) of 4′-aminoacetophenone, 1.6 g (18 mmol, 5.0 eq.) of methacrylic acid, and 0.25 mL of 1,3-dimethyl-2-imidazolidinone (DMI) as a solvent were mixed in a 2 mL vial for microwave reaction, 70 mg (0.37 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 250° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.8/1.0 (molar ratio).

### Example 12

5 mg of 4-hydroxy TEMPO, 0.5 g (3.9 mmol, 1.0 eq.) of 4-chloroaniline, 1.7 g (20 mmol, 5.0 eq.) of methacrylic acid, and 0.25 mL of 1,3-dimethyl-2-imidazolidinone (DMI) as a solvent were mixed in a 2 mL vial for microwave reaction, 74 mg (0.39 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 250° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=1.4/1.0 (molar ratio).

### Example 13

5 mg of 4-hydroxy TEMPO, 0.5 g (4.0 mmol, 1.0 eq.) of 4-fluoro-2-methylaniline, 1.7 g (20 mmol, 5.0 eq.) of methacrylic acid, and 0.25 mL of 1,3-dimethyl-2-imidazolidinone (DMI) as a solvent were mixed in a 2 mL vial for microwave reaction, 76 mg (0.40 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 250° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=4.7/1.0 (molar ratio).

### Comparative Example 1

10 mg of 4-hydroxy TEMPO, 1.0 g (5.8 mmol, 1.0 eq.) of sulfanylamide, and 1.25 g (14.5 mmol, 2.5 eq.) of methacrylic acid were charged into a 2 mL vial for microwave reaction to prepare a reaction mixed solution, and the vial was sealed. The vial was set in a microwave reactor manufactured by Biotage Ltd., and an amidation reaction was carried out by setting reaction conditions as follows. No solvent was used in the reaction system.

<Reaction Conditions>
Set temperature: 115° C.
Pre-stirring: 2 minutes
Reaction time: 5 minutes
Stirring speed: 900 rpm
Absorption level: very high

After completion of the reaction, an internal pressure was released with an injection needle, and then the vial was opened. In a case where the obtained reaction solution was analyzed by NMR, the target 1,2-adduct was not observed, and only the by-product 1,4-adduct was observed.

### Comparative Example 2

5 mg of 4-hydroxy TEMPO, 0.5 g (2.9 mmol, 1.0 eq.) of sulfanylamide, 1.5 g (15 mmol, 5.0 eq.) of methyl methacrylate, and 0.25 mL of 1,3-dimethyl-2-imidazolidinone (DMI) as a solvent were mixed in a 2 mL vial for microwave reaction, 55 mg (0.29 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 115° C.
Pre-stirring: 2 minutes
Reaction time: 5 minutes
Stirring speed: 900 rpm
Absorption level: very high

After completion of the reaction, an internal pressure was released with an injection needle, and then the vial was opened. In a case where the obtained reaction solution was analyzed by NMR, the target 1,2-adduct was not observed, and only the by-product 1,4-adduct was observed.

### Comparative Example 3

5 mg of 4-hydroxy TEMPO, 0.5 g (2.9 mmol, 1.0 eq.) of sulfanylamide, 2.4 g (15 mmol, 5.0 eq.) of phenyl methacrylate, and 0.25 mL of 1,3-dimethyl-2-imidazolidinone (DMI) as a solvent were mixed in a 2 mL vial for microwave reaction, 55 mg (0.29 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 115° C.
Pre-stirring: 2 minutes
Reaction time: 5 minutes
Stirring speed: 900 rpm
Absorption level: very high

After completion of the reaction, an internal pressure was released with an injection needle, and then the vial was opened. In a case where the obtained reaction solution was analyzed by NMR, the target 1,2-adduct was not observed, and only the by-product 1,4-adduct was observed.

### Comparative Example 4

5 mg of 4-hydroxy TEMPO, 0.5 g (3.7 mmol, 1.0 eq.) of 4′-aminoacetophenone, 1.6 g (18 mmol, 5.0 eq.) of methacrylic acid, and 0.25 mL of 1,3-dimethyl-2-imidazolidinone (DMI) as a solvent were mixed in a 2 mL vial for microwave reaction, 70 mg (0.37 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 115° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.012/1.0 (molar ratio).

### Comparative Example 5

5 mg of 4-hydroxy TEMPO, 0.5 g (3.9 mmol, 1.0 eq.) of 4-chloroaniline, 1.7 g (20 mmol, 5.0 eq.) of methacrylic acid, and 0.25 mL of 1,3-dimethyl-2-imidazolidinone (DMI) as a solvent were mixed in a 2 mL vial for microwave reaction, 74 mg (0.39 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 115° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.063/1.0 (molar ratio).

### Comparative Example 6

5 mg of 4-hydroxy TEMPO, 0.5 g (4.0 mmol, 1.0 eq.) of 4-fluoro-2-methylaniline, 1.7 g (20 mmol, 5.0 eq.) of methacrylic acid, and 0.25 mL of 1,3-dimethyl-2-imidazolidinone (DMI) as a solvent were mixed in a 2 mL vial for microwave reaction, 76 mg (0.40 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 115° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.12/1.0 (molar ratio).

### Comparative Example 7

5 mg of 4-hydroxy TEMPO, 0.5 g (2.5 mmol, 1.0 eq.) of 2-aminobenzhydrol, and 1.1 g (13 mmol, 5.0 eq.) of methacrylic acid were charged into a 2 mL vial for microwave reaction to prepare a reaction mixed solution, and the vial was sealed. The vial was set in a microwave reactor manufactured by Biotage Ltd., and an amidation reaction was carried out by setting reaction conditions as follows.
Set temperature: 250° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, due to the large amount of by-products, the ratio of the 1,2-adduct to the 1,4-adduct could not be determined.

### Example 14

5 mg of 4-hydroxy TEMPO, 0.5 g (2.9 mmol, 1.0 eq.) of sulfanylamide, 0.62 g (7.3 mmol, 2.5 eq.) of methacrylic acid, and 0.25 mL of N-methylaniline (NMP) as a solvent were mixed in a 2 mL vial for microwave reaction, 42 mg (0.29 mmol, 0.1 eq.) of diphosphorus pentoxide as a phosphorus oxide compound catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 200° C.
Pre-stirring: 0.5 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=2.1/1.0 (molar ratio).

### Example 15

5 mg of 4-hydroxy TEMPO, 0.5 g (2.9 mmol, 1.0 eq.) of sulfanylamide, 0.62 g (7.3 mmol, 2.5 eq.) of methacrylic acid, and 0.25 mL of N-methylaniline (NMP) as a solvent were mixed in a 2 mL vial for microwave reaction, 0.21 g (1.45 mmol, 0.5 eq.) of diphosphorus pentoxide as a phosphorus oxide compound catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 200° C.
Pre-stirring: 0.5 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=72/1.0 (molar ratio).

### Example 16

5 mg of 4-hydroxy TEMPO, 0.5 g (2.9 mmol, 1.0 eq.) of sulfanylamide, 0.62 g (7.3 mmol, 2.5 eq.) of methacrylic acid, and 0.25 mL of N-methylaniline (NMP) as a solvent were mixed in a 2 mL vial for microwave reaction, 0.17 g (1.2 mmol, 0.4 eq.) of diphosphorus pentoxide as a phosphorus oxide compound catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 200° C.
Pre-stirring: 0.5 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=3.9/1.0 (molar ratio).

2 mL of acetonitrile/water=1/2 (molar ratio) was added to the vial after the completion of the reaction, and the mixture was stirred at room temperature for 30 minutes. The obtained solid was collected by suction filtration, and washed with 1 mL of acetonitrile/water=1/2 (molar ratio). The obtained solid was dried under reduced pressure to obtain 0.50 g of a target 1,2-adduct at a yield of 70%.

### Example 17

5 mg of 4-hydroxy TEMPO, 0.5 g (2.9 mmol, 1.0 eq.) of sulfanylamide, 0.26 g (3.0 mmol, 1.05 eq.) of methacrylic acid, and 0.25 mL of N-methylaniline (NMP) as a solvent were mixed in a 2 mL vial for microwave reaction, 0.17 g (1.2 mmol, 0.4 eq.) of diphosphorus pentoxide as a phosphorus oxide compound catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 200° C.
Pre-stirring: 0.5 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=10/1.0 (molar ratio).

### Example 18

5 mg of 4-hydroxy TEMPO, 0.5 g (2.9 mmol, 1.0 eq.) of sulfanylamide, 0.26 g (3.0 mmol, 1.05 eq.) of methacrylic acid, and 0.25 mL of N-methylaniline (NMP) as a solvent were mixed in a 2 mL vial for microwave reaction, 0.17 g (1.2 mmol, 0.4 eq.) of diphosphorus pentoxide as a phosphorus oxide compound catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 225° C.
Pre-stirring: 0.5 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=12/1.0 (molar ratio).

### Example 19

5 mg of 4-hydroxy TEMPO, 0.5 g (2.9 mmol, 1.0 eq.) of sulfanylamide, 0.26 g (3.0 mmol, 1.05 eq.) of methacrylic acid, and 0.25 mL of N-methylaniline (NMP) as a solvent were mixed in a 2 mL vial for microwave reaction, 0.17 g (1.2 mmol, 0.4 eq.) of diphosphorus pentoxide as a phosphorus oxide compound catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 150° C.
Pre-stirring: 0.5 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=11/1.0 (molar ratio).

### Example 20

5 mg of 4-hydroxy TEMPO, 0.5 g (2.9 mmol, 1.0 eq.) of sulfanylamide, 0.26 g (3.0 mmol, 1.05 eq.) of methacrylic acid, and 0.25 mL of N-methylaniline (NMP) as a solvent were mixed in a 2 mL vial for microwave reaction, and 0.17 g (1.2 mmol, 0.4 eq.) of diphosphorus pentoxide as a phosphorus oxide compound catalyst was added thereto to prepare a reaction mixed solution. The vial was sealed, and then heated and stirred in an oil bath at 225° C. for 10 minutes.

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=10/1.0 (molar ratio).

### Example 21

5 mg of 4-hydroxy TEMPO, 0.5 g (2.9 mmol, 1.0 eq.) of sulfanylamide, 1.2 g (15 mmol, 5.0 eq.) of methacrylic acid, and 0.25 mL of 1,3-dimethyl-2-imidazolidinone (DMI) as a solvent were mixed in a 2 mL vial for microwave reaction, 28 mg (0.3 mmol, 0.1 eq.) of methanesulfonic acid as a Broensted acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 250° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=4.0/1.0 (molar ratio).

### Example 22

5 mg of 4-hydroxy TEMPO, 0.5 g (2.9 mmol, 1.0 eq.) of sulfanylamide, 1.2 g (15 mmol, 5.0 eq.) of methacrylic acid, and 0.25 mL of xylene as a solvent were mixed in a 2 mL vial for microwave reaction, 28 mg (0.3 mmol, 0.1 eq.) of methanesulfonic acid as a Broensted acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 250° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=13/1.0 (molar ratio).

### Example 23

10 mg of 4-hydroxy TEMPO, 1.0 g (5.8 mmol, 1.0 eq.) of sulfanylamide, and 1.25 g (14.5 mmol, 2.5 eq.) of methacrylic acid were charged into a 2 mL vial for microwave reaction to prepare a reaction mixed solution, and the vial was sealed. The vial was set in a microwave reactor manufactured by Biotage Ltd., and an amidation reaction was carried out by setting reaction conditions as follows. No solvent was used in the reaction system.

<Reaction Conditions>
Set temperature: 125° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.04/1.0 (molar ratio).

### Example 24

5 mg of 4-hydroxy TEMPO, 0.5 g (2.9 mmol, 1.0 eq.) of sulfanylamide, 1.5 g (15 mmol, 5.0 eq.) of methyl methacrylate, and 0.25 mL of 1,3-dimethyl-2-imidazolidinone (DMI) as a solvent were mixed in a 2 mL vial for microwave reaction, 55 mg (0.29 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 125° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.23/1.0 (molar ratio).

### Example 25

5 mg of 4-hydroxy TEMPO, 0.5 g (2.9 mmol, 1.0 eq.) of sulfanylamide, 2.4 g (15 mmol, 5.0 eq.) of phenyl methacrylate, and 0.25 mL of 1,3-dimethyl-2-imidazolidinone (DMI) as a solvent were mixed in a 2 mL vial for microwave reaction, 55 mg (0.29 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 125° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.25/1.0 (molar ratio).

### Example 26

5 mg of 4-hydroxy TEMPO, 0.5 g (3.7 mmol, 1.0 eq.) of 4′-aminoacetophenone, 1.6 g (18 mmol, 5.0 eq.) of methacrylic acid, and 0.25 mL of 1,3-dimethyl-2-imidazolidinone (DMI) as a solvent were mixed in a 2 mL vial for microwave reaction, 70 mg (0.37 mmol, 0.1 eq.) of titanium tetrachloride as a Lewis acid catalyst was added thereto to prepare a reaction mixed solution, and the vial was sealed. The vial was set in a microwave reactor manufactured by Biotage Ltd., and an amidation reaction was carried out by setting reaction conditions as follows.
Set temperature: 125° C.
Pre-stirring: 2 minutes
Reaction time: 5 minutes
Stirring speed: 900 rpm
Absorption level: very high

After completion of the reaction, an internal pressure was released with an injection needle, and then the vial was opened. The obtained reaction solution was analyzed by NMR, and from the comparison of integrated values, a ratio between an amount of target 1,2-adduct produced and an amount of by-product 1,4-adduct produced was obtained. As a result, [1,2-adduct]/[1,4-adduct]=0.1/1.0 (molar ratio).

### Example 27

50.0 mg of 4-hydroxy TEMPO, 10.00 g (58.07 mmol, 1.0 eq.) of sulfanylamide, 8.75 g (101.63 mmol, 1.75 eq.) of methacrylic acid, and 20 mL of N-methylpyrrolidone (NMP) as a solvent were charged into a 300 mL three-neck flask, and mixed at 80° C. After purging an inside of a test tube with nitrogen, 5.95 g (40.65 mmol, 0.7 eq.) of phosphorus pentoxide was added thereto, and the mixture was heated and stirred at 125° C. for 4 hours. After completion of the reaction, 30 mL of water/methanol=9/1 (volume ratio) was added thereto, and the mixture was stirred at 40° C. for 30 minutes and stirred at 0° C. for 30 minutes. The precipitated solid was suction-filtered and washed twice with 20 mL of water/methanol=9/1 (volume ratio). The obtained solid was dried under reduced pressure at 40° C. for 2 hours to obtain 11.7 g of 4-sulfamoylphenyl methacrylamide which was the target 1,2-adduct (yield: 84%).

### Example 28

5.0 mg of 4-hydroxy TEMPO, 1.0 g (7.24 mmol, 1.0 eq.) of 4-nitroaniline, 1.09 g (12.67 mmol, 1.75 eq.) of methacrylic acid, and 2.0 mL of N-methylpyrrolidone (NMP) as a solvent were charged and mixed in a 30 mL test tube. After purging an inside of the test tube with nitrogen, 0.74 g (5.1 mmol, 0.7 eq.) of phosphorus pentoxide was added thereto, and the mixture was heated and stirred at 125° C. for 4 hours.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 76.44%, and the area percentage of the by-product 1,4-adduct was 2.24%.

### Example 29

5.0 mg of 4-hydroxy TEMPO, 1.0 g (7.75 mmol, 1.0 eq.) of 2,4-difluoroaniline, 1.16 g (13.55 mmol, 1.75 eq.) of methacrylic acid, and 2.0 mL of N-methylpyrrolidone (NMP) as a solvent were charged and mixed in a 30 mL test tube. After purging an inside of the test tube with nitrogen, 0.79 g (5.42 mmol, 0.7 eq.) of phosphorus pentoxide was added thereto, and the mixture was heated and stirred at 125° C. for 4 hours.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 95.90%, and the area percentage of the by-product 1,4-adduct was 0.29%.

### Example 30

5.0 mg of 4-hydroxy TEMPO, 1.0 g (9.00 mmol, 1.0 eq.) of 2-fluoroaniline, 1.36 g (15.75 mmol, 1.75 eq.) of methacrylic acid, and 2.0 mL of N-methylpyrrolidone (NMP) as a solvent were charged and mixed in a 30 mL test tube. After purging an inside of the test tube with nitrogen, 0.92 g (6.30 mmol, 0.7 eq.) of phosphorus pentoxide was added thereto, and the mixture was heated and stirred at 125° C. for 4 hours.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 92.95%, and the area percentage of the by-product 1,4-adduct was 1.20%.

### Example 31

5.0 mg of 4-hydroxy TEMPO, 1.0 g (7.99 mmol, 1.0 eq.) of 4-fluoro-2-methylaniline, 1.20 g (13.98 mmol, 1.75 eq.) of methacrylic acid, and 2.0 mL of N-methylpyrrolidone (NMP) as a solvent were charged and mixed in a 30 mL test tube. After purging an inside of the test tube with nitrogen, 0.82 g (5.59 mmol, 0.7 eq.) of phosphorus pentoxide was added thereto, and the mixture was heated and stirred at 125° C. for 4 hours.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 77.17%, and the area percentage of the by-product 1,4-adduct was 1.39%.

### Example 32

5.0 mg of 4-hydroxy TEMPO, 1.0 g (6.21 mmol, 1.0 eq.) of 4-(fluoromethyl)aniline, 0.94 g (10.86 mmol, 1.75 eq.) of methacrylic acid, and 2.0 mL of N-methylpyrrolidone (NMP) as a solvent were charged and mixed in a 30 mL test tube. After purging an inside of the test tube with nitrogen, 0.64 g (4.34 mmol, 0.7 eq.) of phosphorus pentoxide was added thereto, and the mixture was heated and stirred at 125° C. for 4 hours.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 70.59%, and the by-product 1,4-adduct was not observed.

### Example 33

5.0 mg of 4-hydroxy TEMPO, 1.0 g (7.84 mmol, 1.0 eq.) of 4-chloroaniline, 1.18 g (13.72 mmol, 1.75 eq.) of methacrylic acid, and 2.0 mL of N-methylpyrrolidone (NMP) as a solvent were charged and mixed in a 30 mL test tube. After purging an inside of the test tube with nitrogen, 0.80 g (5.49 mmol, 0.7 eq.) of phosphorus pentoxide was added thereto, and the mixture was heated and stirred at 125° C. for 4 hours.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 71.20%, and the area percentage of the by-product 1,4-adduct was 2.02%.

### Example 34

5.0 mg of 4-hydroxy TEMPO, 1.0 g (7.40 mmol, 1.0 eq.) of 4-acetylaniline, 1.11 g (12.95 mmol, 1.75 eq.) of methacrylic acid, and 2.0 mL of N-methylpyrrolidone (NMP) as a solvent were charged and mixed in a 30 mL test tube. After purging an inside of the test tube with nitrogen, 0.76 g (5.18 mmol, 0.7 eq.) of phosphorus pentoxide was added thereto, and the mixture was heated and stirred at 125° C. for 4 hours.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 22.86%, and the area percentage of the by-product 1,4-adduct was 11.39%.

### Example 35

5.0 mg of 4-hydroxy TEMPO, 1.0 g (4.78 mmol, 1.0 eq.) of 3,5-dimethoxycarbonylaniline, 0.72 g (8.37 mmol, 1.75 eq.) of methacrylic acid, and 2.0 mL of N-methylpyrrolidone (NMP) as a solvent were charged and mixed in a 30 mL test tube. After purging an inside of the test tube with nitrogen, 0.49 g (3.35 mmol, 0.7 eq.) of phosphorus pentoxide was added thereto, and the mixture was heated and stirred at 125° C. for 1 hour.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 82.31%, and the by-product 1,4-adduct was not observed.

### Example 36

10 mg of 4-hydroxy TEMPO, 1.0 g (3.12 mmol, 1.0 eq.) of 2,2-bis(trifluoromethyl)benzidine, 0.94 g (10.93 mmol, 3.50 eq.) of methacrylic acid, and 4.0 mL of N-methylpyrrolidone (NMP) as a solvent were charged and mixed in a 30 mL test tube. After purging an inside of the test tube with nitrogen, 0.64 g (4.37 mmol, 1.40 eq.) of phosphorus pentoxide was added thereto, and the mixture was heated and stirred at 125° C. for 1 hour.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 66.08%, and the by-product 1,4-adduct was not observed.

### Example 37

5.0 mg of 4-hydroxy TEMPO, 1.0 g (6.21 mmol, 1.0 eq.) of 4-(fluoromethyl)aniline, 0.94 g (10.86 mmol, 1.75 eq.) of methacrylic acid, and 2.0 mL of diethylene glycol dimethyl ether as a solvent were charged and mixed in a 30 mL test tube. After purging an inside of the test tube with nitrogen, 0.64 g (4.34 mmol, 0.7 eq.) of phosphorus pentoxide was added thereto, and the mixture was heated and stirred at 125° C. for 1 hour.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 85.48%, and the by-product 1,4-adduct was not observed.

### Example 38

5.0 mg of 4-hydroxy TEMPO, 1.0 g (6.62 mmol, 1.0 eq.) of methyl 4-aminobenzoate, 1.00 g (11.58 mmol, 1.75 eq.) of methacrylic acid, and 2.0 mL of N-methylpyrrolidone (NMP) as a solvent were charged and mixed in a 30 mL test tube. After purging an inside of the test tube with nitrogen, 0.68 g (4.63 mmol, 0.70 eq.) of phosphorus pentoxide was added thereto, and the mixture was heated and stirred at 125° C. for 1 hour.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 48.66%, and the area percentage of the by-product 1,4-adduct was 2.13%.

### Example 39

5.0 mg of 4-hydroxy TEMPO, 1.0 g (6.62 mmol, 1.0 eq.) of 4-aminobenzoate, 1.00 g (11.58 mmol, 1.75 eq.) of methacrylic acid, and 2.0 mL of diethylene glycol dimethyl ether as a solvent were charged and mixed in a 30 mL test tube. After purging an inside of the test tube with nitrogen, 0.68 g (4.63 mmol, 0.70 eq.) of phosphorus pentoxide was added thereto, and the mixture was heated and stirred at 125° C. for 1 hour.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 58.40%, and the area percentage of the by-product 1,4-adduct was 1.60%.

### Example 40

5.0 mg of 4-hydroxy TEMPO, 1.0 g (6.62 mmol, 1.0 eq.) of methyl 4-aminobenzoate, 1.00 g (11.58 mmol, 1.75 eq.) of methacrylic acid, and 2.0 mL of sulfolane as a solvent were charged and mixed in a 30 mL test tube. After purging an inside of the test tube with nitrogen, 0.68 g (4.63 mmol, 0.70 eq.) of phosphorus pentoxide was added thereto, and the mixture was heated and stirred at 125° C. for 1 hour.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 41.39%, and the area percentage of the by-product 1,4-adduct was 3.16%.

### Example 41

5.0 mg of 4-hydroxy TEMPO, 1.0 g (6.62 mmol, 1.0 eq.) of methyl 4-aminobenzoate, 1.00 g (11.58 mmol, 1.75 eq.) of methacrylic acid, and 2.0 mL of propylene carbonate as a solvent were charged and mixed in a 30 mL test tube. After purging an inside of the test tube with nitrogen, 0.68 g (4.63 mmol, 0.70 eq.) of phosphorus pentoxide was added thereto, and the mixture was heated and stirred at 125° C. for 1 hour.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 30.83%, and the area percentage of the by-product 1,4-adduct was 1.39%.

### Example 42

5.0 mg of 4-hydroxy TEMPO, 1.0 g (6.62 mmol, 1.0 eq.) of methyl 4-aminobenzoate, 1.00 g (11.58 mmol, 1.75 eq.) of methacrylic acid, and 2.0 mL of 1,3-dimethyl-2-imidazolinone (DMI) as a solvent were charged and mixed in a 30 mL test tube. After purging an inside of the test tube with nitrogen, 0.68 g (4.63 mmol, 0.70 eq.) of phosphorus pentoxide was added thereto, and the mixture was heated and stirred at 125° C. for 1 hour.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 88.48%, and the area percentage of the by-product 1,4-adduct was 3.86%.

### Example 43

5.0 mg of 4-hydroxy TEMPO, 1.0 g (6.21 mmol, 1.0 eq.) of 4-(trifluoromethyl)aniline, 0.94 g (10.86 mmol, 1.75 eq.) of methacrylic acid, and 2.0 mL of 1,3 -dimethyl-2-imidazolinone (DMI) as a solvent were charged and mixed in a 30 mL test tube. After purging an inside of the test tube with nitrogen, 0.64 g (4.34 mmol, 0.7 eq.) of phosphorus pentoxide was added thereto, and the mixture was heated and stirred at 125° C. for 1 hour.

After completion of the reaction, 10 mg of the obtained reaction solution was diluted with 10 mL of a solvent prepared by mixing acetonitrile/dimethyl sulfoxide at a ratio of 9/1, and the mixture was analyzed by high performance liquid chromatography. As a result, the area percentage of the target 1,2-adduct was 92.12%, and the by-product 1,4-adduct was not observed.

### Example 44

5.0 mg of 4-hydroxy TEMPO, 1.0 g (7.75 mmol, 1.0 eq.) of 2,4-difluoroaniline, 0.98 g (13.55 mmol, 1.75 eq.) of acrylic acid, and 2.0 mL of N-methylpyrrolidone (NMP) as a solvent were charged and mixed in a 30 mL test tube. After purging an inside of the test tube with nitrogen, 0.79 g (5.42 mmol, 0.7 eq.) of phosphorus pentoxide was added thereto, and the mixture was heated and stirred at 125° C. for 1 hour.

After completion of the reaction, 10 mg of the obtained reaction solution was diluted with 10 mL of a solvent prepared by mixing acetonitrile/dimethyl sulfoxide at a volume ratio of 9/1, and the mixture was analyzed by high performance liquid chromatography. As a result, the area percentage of the target 1,2-adduct was 77.91%, and the by-product 1,4-adduct was not observed.

### Example 45

5.0 mg of 4-hydroxy TEMPO, 1.0 g (7.75 mmol, 1.0 eq.) of 2,4-difluoroaniline, 0.98 g (13.55 mmol, 1.75 eq.) of acrylic acid, and 2.0 mL of 1,3-dimethyl-2-imidazolinone (DMI) as a solvent were charged and mixed in a 30 mL test tube. After purging an inside of the test tube with nitrogen, 0.79 g (5.42 mmol, 0.7 eq.) of phosphorus pentoxide was added thereto, and the mixture was heated and stirred at 125° C. for 1 hour.

After completion of the reaction, 10 mg of the obtained reaction solution was diluted with 10 mL of a solvent prepared by mixing acetonitrile/dimethyl sulfoxide at a ratio of 9/1, and the mixture was analyzed by high performance liquid chromatography. As a result, the area percentage of the target 1,2-adduct was 71.42%, and the by-product 1,4-adduct was not observed.

The present invention has been described with the embodiments thereof, any details of the description of the present invention are not limited unless described otherwise, and it is obvious that the present invention is widely construed without departing from the gist and scope of the present invention described in the accompanying claims.

What is claimed is:

1. A method for producing an N-(hetero)aryl (meth)acrylamide compound, the production method comprising:
reacting a compound represented by General Formula (1) with a compound represented by General Formula (2) at a temperature higher than 120° C. to carry out amidation and to obtain a compound represented by General Formula (3), 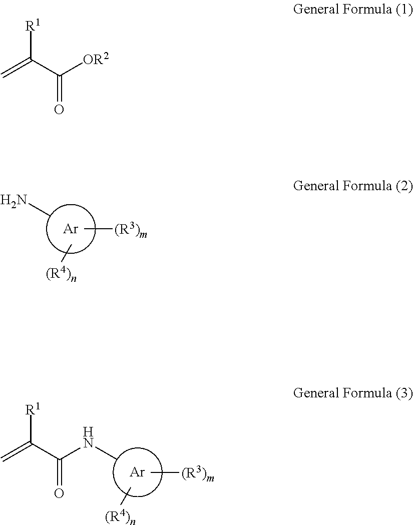
in each formula, R¹ represents a hydrogen atom or an aliphatic group, R² represents a hydrogen atom, a chain-like aliphatic group, an aliphatic hydrocarbon ring group, an aryl group, or a heterocyclic group, Ar represents an aromatic ring, R³ represents an electron withdrawing group and m is an integer of 1 or more, R⁴ represents a chain-like aliphatic group, an aliphatic hydrocarbon ring group, an aryl group, or a heterocyclic group, and n is an integer of 0 or more, where R⁴ is not an α-hydroxybenzyl group, and a maximum value of m+n is a maximum value of the number of substituents which can be included in ring-constituting atoms of Ar.

2. The production method according to claim 1,
wherein the amidation reaction is carried out in a presence of at least one reaction catalyst.

3. The production method according to claim 2,
wherein the reaction catalyst is at least one of a Lewis acid, a Broensted acid, a metal oxide, or a phosphorus oxide compound.

4. The production method according to claim 2,
wherein at least one of a Lewis acid, a Broensted acid, or a phosphorus oxide compound is used as the reaction catalyst.

5. The production method according to claim 1,
wherein a reaction temperature of the amidation reaction is set to 130° C. or higher.

6. The production method according to claim 5,
wherein the reaction temperature of the amidation reaction is set to 140° C. or higher.

7. The production method according to claim 1,
wherein Ar represents a benzene ring.

8. The production method according to claim 7,
wherein R¹ represents a hydrogen atom or an alkyl group having 1 to 5 carbon atoms.

9. The production method according to claim 7,
wherein m is an integer of 1 to 3 and n is an integer of 0 to 4.

10. The production method according to claim 1,
wherein a reaction temperature of the amidation reaction is controlled by microwave irradiation.

11. The production method according to claim 1,
wherein the amidation reaction is carried out by a flow-type reaction.
